(19) United States
(12) Patent Application Publication
(10) Pub. No.: US 2025/0035628 A1
(43) Pub. Date: Jan. 30, 2025

(54) BIOASSAYS TO MEASURE THE SYNERGISTIC ANTIBODY-DEPENDENT ENHANCEMENT (ADE) EFFECT OF SARS-CoV-2 NEUTRALIZING ANTIBODIES

(71) Applicant: WuXi Biologics Ireland Limited, Louth (IE)

(72) Inventors: Min Chen, Shanghai (CN); Xiaoqing Jia, Shanghai (CN)

(21) Appl. No.: 18/716,648

(22) PCT Filed: Nov. 30, 2022
(86) PCT No.: PCT/CN2022/135470

§ 371 Date: Jun. 5, 2024

(30) Foreign Application Priority Data
Dec. 6, 2021 (WO) PCT/CN2021/135840

(51) Int. Cl. G01N 33/569; G01N 33/50

(52) U.S. Cl. CPC G01N 33/56983; G01N 33/5035; G01N 33/5044; G01N 2333/165

(57) ABSTRACT

Provided herein are bioassays to measure the synergistic antibody-dependent enhancement (ADE) effect of SARS-CoV-2 neutralizing antibodies. In particular, the disclosure relates to a method for determining whether two or more SARS-CoV-2 neutralizing antibodies have synergistic ADE effect comprising contacting a combination of said two or more SARS-CoV-2 neutralizing antibodies with FcγR-expressing cells infected with a pseudovirus pseudotyped with SARS-CoV-2 antigen; and determining whether the combination increases the internalization of the pseudovirus into the FcγR-expressing cells synergistically.

### TECHNICAL FIELD

The present disclosure concerns the field of biopharm analytics and clinical diagnosis and therapy. It inter alia pertains to bioassays to measure the synergistic antibody-dependent enhancement (ADE) effect of SARS-CoV-2 neutralizing antibodies.

### BACKGROUND

The coronavirus disease-2019 (COVID-19) pandemic, caused by severe acute respiratory syndrome coronavirus-2 (SARS-CoV-2), represents an unprecedented global public health emergency with economic and social consequences. Any means to improve the condition of patients, accelerate their recovery, and reduce the risk of deterioration and death would be considered of significant clinical and economic importance.

With respect to the immune response generated by the host, the specific neutralizing antibodies generated against the virus are considered essential in the control of virus infections in various ways. However, in some cases, the presence of specific antibodies can be beneficial for the virus. This activity known as antibody-dependent enhancement (ADE) of virus infection enhances virus entry and in some cases virus replication into host cells through interaction with Fc and/or complement receptors. It has been also reported in data from previous CoV research studies that ADE may play a role in the virus's pathology (Gabriela Athziri Sánchez-Zuno et al. (2021), “A review: Antibody-dependent enhancement in COVID-19: The not so friendly side of antibodies”, International Journal of Immunopathology and Pharmacology, Volume 35: 1-15).

Antibody-dependent Enhancement (ADE) refers to a phenomenon by which antiviral antibodies enhance the entry and replication of virus into immune cells by the FcγR pathway (Tirado S M, Yoon K J (2003). “Antibody-dependent enhancement of virus infection and disease”. Viral Immunology. 16 (1): 69-86.). This phenomenon has been reported in vitro and in vivo for viruses representing numerous families and genera of public health and veterinary importance. These viruses share some common features such as preferential replication in macrophages, ability to establish persistence, and antigenic diversity.

ADE can be an either protective or pathogenic effect of the antiviral antibodies: if the antibody-opsonized virions and infected cells are eliminated by phagocytosis, cytotoxicity, and induction of adaptive immune responses, it is protective (Bournazos S, DiLillo D J, Ravetch J V (2015). The role of Fc-Fcγ, interactions in IgG-mediated microbial neutralization. J. Exp. Med. 212, 1361-1369).

However, antibody binding of the virus can also cause viral entry of the FcγR bearing immune cells, viral replication, and enhanced infection and virulence (Iwasaki A, Yang Y (2020). The potential danger of suboptimal antibody responses in COVID-19. Nature Reviews. Immunology. 20 (6): 339-341).

Hence, the occurrence of ADE may represent one of the greatest challenges for scientists working on the development of a safe vaccine against COVID-19. Even though several vaccines have been approved from regulatory bodies under emergency conditions and are distributed worldwide, we cannot rule out the possibility that the evolution of the virus can directly affect its targets, and therefore, finding and assessing new antibodies candidates with high efficiency and safety in neutralizing the virus is very important.

If the vaccines are not capable of generating neutralizing antibodies against the possible mutagenic variants to mount a response, the result may lead to the generation of sub-neutralizing antibodies that will even be capable of facilitating uptake by macrophages that express FcR, with the subsequent stimulation of macrophages and production of pro-inflammatory cytokines.

ADE of SARS-CoV-2 infection and immunity have a role in the disease development and therapeutic endpoints. Synergy of ADE can be pivotal biological endpoints that two or more neutralizing antibodies should be used together or not. It can also help a clinician to determine the clinical outcome by testing the synergetic ADE effect using serum neutralizing antibodies from COVID-19 patients. However, there have been no bioassays to assess the synergistic effect of anti-SARS-CoV-2 antibodies.

Hence, there is a great need for bioassays to measure the antibody-dependent enhancement (ADE) effect of SARS-CoV-2 neutralizing antibodies, especially the synergic effects of two or more SARS-CoV-2 neutralizing antibodies.

### SUMMARY OF THE INVENTION

Here, we developed in vitro bioassays enabling the assessment of synergistic antibody-dependent enhancement effect and its control mechanism of the anti-SARS-CoV-2 neutralizing antibodies through analysis of FcγR-expressing cell lines infected with a luciferase-expressing vesicular stomatitis virus (VSV) pseudotyped with SARS-CoV-2 spike protein.

According to a first aspect, disclosed herein is a method for determining whether two or more antibodies (such as SARS-CoV-2 neutralizing antibodies) have synergistic antibody-dependent enhancement (ADE) effect in SARS-COV-2 infection, wherein the method comprises: contacting a combination of said two or more antibodies with FcR-expressing cells infected with pseudovirus, wherein the pseudovirus is pseudotyped with SARS-CoV-2 antigen; and determining whether the combination increases the internalization of the pseudovirus into the FcR-expressing cells synergistically.

In some embodiments, the FcR is a FcγR. In some embodiments, the FcγR is selected from FcγRI (e.g., FcγRIa, FcγRIb and FcγRIc), FcγRII (e.g., FcγRIIa, FcγRIIb and FcγRIIc) and FcγRII (e.g., FcγRIIIa or FcγRIIIb), preferably FcγRII (such as FcγRIIa).

In some embodiments, the FcR-expressing cell is selected from the group consisting of natural FcR-expressing cells, such as monocytes, macrophages, heratopoietic cell; or engineered cell, such as CHO cells, HEK 293 T cells.

In some embodiments, the pseudovirus is selected from the group consisting of VSV; retrovirus system, such as murine leukemia virus (MLV); lentivirus system, such as HIV-1; adenovirus; or adeno-associated virus (AAV).

In some embodiments, the cell further comprises one or more report genes selected from the group consisting of fluorescin (such as luciferase, GFP), β-galactosidase, secreted alkaline phosphatase (SEAP).

In some embodiments, the SARS-CoV-2 antigen is derived from the spike protein (S protein), preferably immunodominant epitopes, such as from the RBD region or the N-terminal domain of S protein.

In some embodiments, the method further comprises the step of determining the binding effect of a combination of the two or more antibodies to FcR (preferably FcγRIIA), such as by ELISA.

In some embodiments, the method further comprises the step of determining the pseudovirus neutralization effect of the combination of the two or more antibodies, such as two or more SARS-CoV-2 neutralizing antibodies.

In some embodiments, the SARS-CoV-2 antigen is derived from S protein and the pseudovirus neutralization effect is tested with ACE2-expressing cells, such as ACE2-expressing CHO-K1 cells.

In some embodiments, the method is used in the selection of antibodies for used in the preparation of a vaccine or a combination of vaccines for preventing SARS-CoV-2 infection.

In some embodiments, the method is used in predicting and/or preventing a risk of ADE effect in the treatment or vaccination for SARS-CoV-2 infection.

In some embodiments, the method is used in the quality control during biopharmaceutical product development, manufacturing and/or storage.

In another aspect, provided herein is a use of substance(s) for determining whether two or more antibodies (such as SARS-CoV-2 neutralizing antibodies) have synergistic antibody-dependent enhancement (ADE) effect in SARS-COV-2 infection in the preparation of a product (such as a kit) used in the selection of antibodies for used in the preparation of a vaccine or a combination of vaccines for preventing SARS-CoV-2 infection.

In another aspect, provided herein is a use of substance(s) for determining whether two or more antibodies (such as SARS-CoV-2 neutralizing antibodies) have synergistic antibody-dependent enhancement (ADE) effect in SARS-COV-2 infection in the preparation of a product (such as a kit) used in the selection of antibodies for used in predicting and/or preventing a risk of ADE effect in the treatment or vaccination for SARS-CoV-2 infection.

In another aspect, provided herein is a use of substance(s) for determining whether two or more antibodies (such as SARS-CoV-2 neutralizing antibodies) have synergistic antibody-dependent enhancement (ADE) effect in SARS-COV-2 infection in the preparation of a product (such as a kit) used in the selection of antibodies for used in the quality control during biopharmaceutical product development, manufacturing and/or storage.

In another aspect, provided herein is a product comprising substance(s) for determining whether two or more antibodies (such as SARS-CoV-2 neutralizing antibodies) have synergistic antibody-dependent enhancement (ADE) effect in SARS-COV-2 infection for use in the selection of antibodies for used in the preparation of a vaccine or a combination of vaccines for preventing SARS-CoV-2 infection.

In another aspect, provided herein is a product comprising substance(s) for determining whether two or more antibodies (such as SARS-CoV-2 neutralizing antibodies) have synergistic antibody-dependent enhancement (ADE) effect in SARS-COV-2 infection for use in the selection of antibodies for used in predicting and/or preventing a risk of ADE effect in the treatment or vaccination for SARS-CoV-2 infection.

In another aspect, provided herein is a product comprising substance(s) for determining whether two or more antibodies (such as SARS-CoV-2 neutralizing antibodies) have synergistic antibody-dependent enhancement (ADE) effect in SARS-COV-2 infection for use in the selection of antibodies for used in the quality control during biopharmaceutical product development, manufacturing and/or storage.

In another aspect, provided herein is a system for determining whether two or more antibodies (such as SARS-CoV-2 neutralizing antibodies) have synergistic antibody-dependent enhancement (ADE) effect in SARS-COV-2 infection, wherein the system comprises means for carrying out the steps in the method of the present application.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

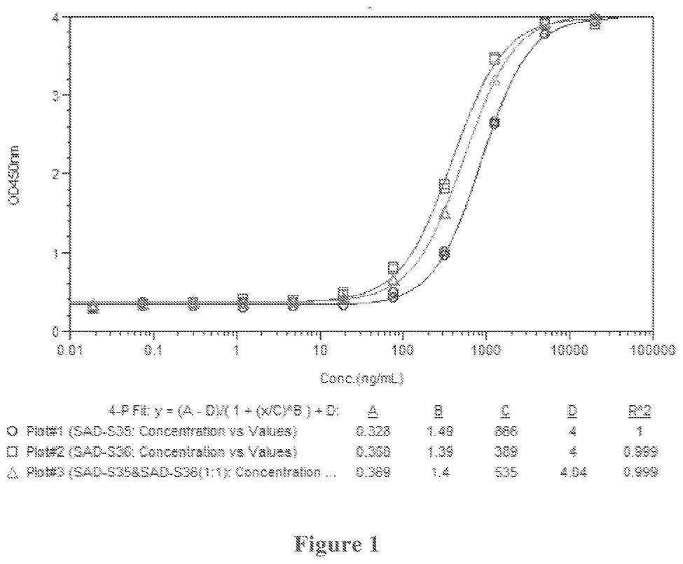

FIG. 1: binding of anti-SARS-CoV-2 RBD neutralizing antibodies to FcγRIIA expressed by CHO-K1 cells infected with a luciferase-expressing vesicular stomatitis virus (VSV) pseudotyped with SARS-CoV-2 spike protein:
Plot #1: SAD-S35 antibody;
Plot #2: SA2-S36 antibody;
Plot #3: SAD-S35 antibody+SA2-S36 antibody (1:1).

4-P Fit is calculated by the following formula:

$$y = D + \frac{A - D}{1 + {( \frac{x}{C} )}^{B}}$$

wherein x=the independent variable; A=Left asymptote; B=curvature hill slope; C=EC₅₀, ng/mL; and D=Right asymptote.

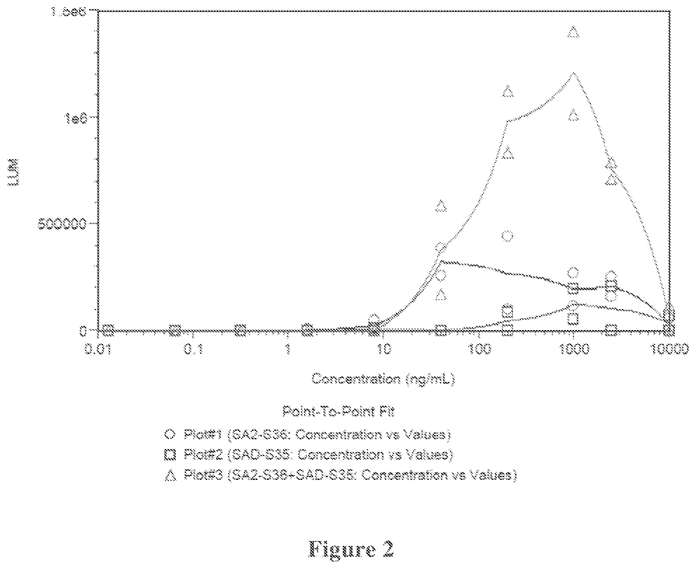

FIG. 2: binding of anti-SARS-CoV-2 RBD neutralizing antibodies to FcγRIIA determined by ELISA:
Plot #1: SA2-S36 antibody;
Plot #2: SAD-S35 antibody;
Plot #3: SA2-S36 antibody+SAD-S35 antibody (1:1).

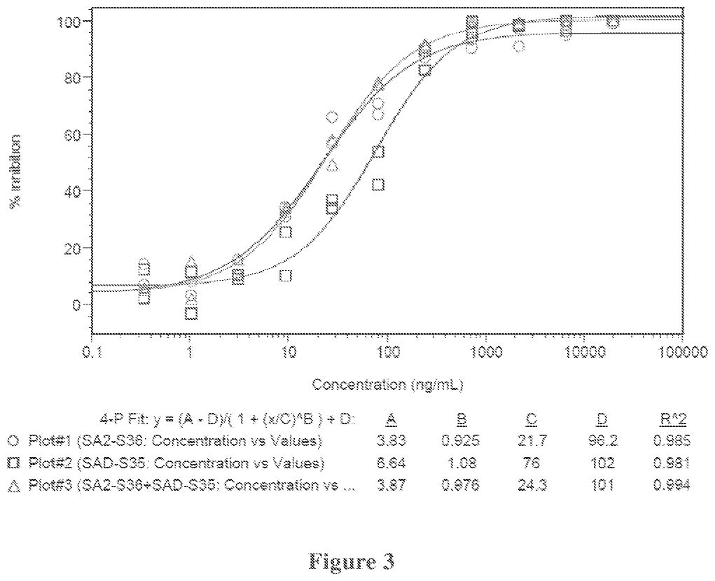

FIG. 3: neutralization effect of anti-SARS-CoV-2 RBD neutralizing antibodies to ACE2-expressing target cells infected with a luciferase-expressing vesicular stomatitis virus (VSV) pseudotyped with SARS-CoV-2 spike protein:
Plot #1: SA2-S36 antibody;
Plot #2: SAD-S35 antibody;
Plot #3: SA2-S36 antibody+SAD-S35 antibody (1:1).

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

The present disclosure is inter alia based on the special in vitro bioassays developed which enables the assessment of synergistic antibody-dependent enhancement effect and its control mechanism of the anti-SARS-CoV-2 neutralizing antibodies through analysis of FcγR-expressing cell lines infected with a luciferase-expressing vesicular stomatitis virus pseudotyped with SARS-CoV-2 spike protein.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that other elements may be present.

The term “isolated” refers to a material that is substantially or essentially free from components that normally accompany it in its native state. The material can be a cell or a macromolecule such as a protein or nucleic acid. For example, an “isolated cell,” as used herein, refers to a cell, which has been purified from the cells in a naturally-occurring state.

Method for determining the synergistic ADE effect Provided herein is a method for determining whether two or more antibodies have synergistic antibody-dependent enhancement (ADE) effect in SARS-CoV-2 infection, wherein the method comprises: contacting a combination of said two or more antibodies with FcR-expressing cells infected with pseudovirus, wherein the pseudovirus is pseudotyped with SARS-CoV-2 antigen; and determining whether the combination increases the internalization of the pseudovirus into the FcR-expressing cells synergistically.

The antibodies to be assessed can be any antibody that may be suspected to produce ADE effect in SARS-CoV-2 infection. For example, an ADE effect may be produced by SARS-CoV-2 neutralizing antibodies, SARS-CoV-2 binding antibodies, antibodies for other coronavirus (such as SARS-CoV-1, MERS-CoV). The antibodies can be monoclonal (such as recombinantly produced or produced by hybridoma or synthesis) or polyclonal, such as derived from SARS-CoV-2 infected subject, especially from a recovered subject. According to the need in practice, the combination of the antibodies may comprise two or more antibodies. In some embodiments, the antibodies are SARS-CoV-2 neutralizing antibodies, such as FcγR binding SARS-CoV-2 neutralizing antibodies.

Without wishing to be bound by theory, it is believed that in the SARS-CoV-2 infection. ADE is mediated by the binding of FcRs, mainly CD32 expressed in different immune cells, including monocytes, macrophages, and B cells; and pre-existing or co-existing CoV-specific antibodies are capable of promoting viral entry into FcR-expressing cells.

In some embodiments, among the FcγR, three classes of molecules have been defined, varying in structure and affinity for IgG. Exemplary FcγR include FcγRI (e.g., FcγRA, FcγRB and FcγRIC), FcγRII (e.g., FcγRIIA, FcγRIIB and FcγRIIC), and FcγRIII (e.g., FcγRIIIA or FcγRIIIB). FcγRI, present on monocytes and macrophages, is a high affinity IgG receptor. FcγRII and FcγRIII are relatively low affinity receptors and appear to only bind antibody in the form of immune complexes. FcγRII are more widely expressed by hematopoietic cells than are FcγRIII.

FcγR and FcεR couple humoral and cellular immunity by directing the interaction of antibodies with effector cells. These receptors are present on most effector cells of the immune system and mediate phagocytosis, antibody-dependent cell-mediated cytotoxicity, activation of inflammatory cells, and many of the biological sequelae associated with antibody-dependent immunity.

Cells that express FcR (such as FcγR) include, but are not limited to, cells of hematopoietic lineage, including but not limited to, macrophages, monocytes, platelets, neutrophils, eosinophils, mast cells, natural killer cells, basophils, B cells, and DC cells. Macrophages, monocytes and DC are preferred, with DC (including Langerhans cells and other DC precursors) being more preferred. Cells expressing FcγR also include immature thymocytes and certain tumor cell lines.

Pseudovirus are engineered viruses that don't replicate provide a tractable model with lower biosafety level (BSL) clearance for scientists to safely study SARS-CoV-2, including research into vaccine efficacy and emerging variants. By replacing their surface envelope proteins with different envelope proteins of SARS-CoV-2, researchers can glean insights into the ways the pathogen infects cells.

Pseudovirus suitable to be used in the present method comprise but not limited to those derived based on lentivirus, adenovirus, adeno-associated virus, retrovirus, for example, VSV, MLV and HIV. Pseudovirus suitable to be used in the present method is commercially available or can be tailored and prepared according to the need in practice.

The SARS-CoV-2 envelope protein or polypeptide carried by the pseudovirus can be any immunogenic polypeptide from SARS-CoV-2, such as derived from the spike protein (S protein), preferably immunodominant epitopes, for example from the RBD region or the N-terminal domain of S protein.

Since as indicated above, it is believed that in the SARS-CoV-2 infection. ADE is mediated by the binding of FcRs, the present method may further comprise the step of determining the binding effect of the antibody alone and/or the combination of the two or more antibodies to FcR (such as by ELISA).

Moreover, the method may further comprise the step of determining the pseudovirus neutralization effect of the combination of the two or more antibodies.

According to the method of the present disclosure, the following assays are carried out: (a) FcR (CD32A) binding activity of the antibody combination; (b) ADE effect of the antibody combination; and (c) neutralization activity of the antibody combination. The result of assay (a) is indicative of whether the antibody combination binds to FcR which is a basis for ADE. The result of assay (b) is indicative of whether the antibody combination can produce any synergistic effect in ADE. The result of assay (c) is indicative of whether the antibody combination can produce any neutralization effect to target. The three assays form a complete and precise assay system for antibody combinations.

### Use of the Method and Corresponding Products

The method of the present disclosure and the corresponding products (such as a kit for determining the synergistic ADE effect) are useful in laboratory, manufacture and clinic.

ADE of SARS-CoV-2 infection and immunity have a role in the disease development and therapeutic endpoints. In some instances, synergy of ADE can be pivotal biological endpoints that two or more antibodies should be used together or not. It can also help a clinician to determine the clinical outcome by testing the synergetic ADE effect using serum neutralizing antibodies from COVID-19 patients.

The method of the present disclosure can be used but not limited to: (a) in the selection of antibodies for used in the preparation of a vaccine or a combination of vaccines for preventing SARS-CoV-2 infection; and/or (b) in predicting and/or preventing a risk of ADE effect in the treatment or vaccination for SARS-CoV-2 infection; and/or (c) in the quality control during biopharmaceutical product development, manufacturing and/or storage.

ADE can be an either protective or pathogenic effect of the antiviral antibodies. After determining the two or more antibodies have synergic effect in SARS-CoV-2 infection, one may make proper selection to the antibodies to increase the protective effect or avoid the pathogenic effect.

### Exemplary Materials and Steps for Carrying Out the Methods

The following are some Exemplary materials and steps for carrying out the methods. Any methods and materials similar or equivalent to those described herein can be used in the practice or testing of the present disclosure.

### A. Evaluation of the Binding of Anti-SARS-CoV-2 Neutralizing Antibodies to FcγRIIA

To elucidate whether there is synergistic effect when the anti-SARS-CoV-2 antibodies bind to the FcγRIIA, a FcγRIIA binding ELISA may be performed. Briefly. FcγRIIA were coated onto the 96-well plate. Following incubation, the plates are washed and blocked. Increasing concentrations (e.g., 0˜20 μg/mL) of the anti-SARS-CoV-2 neutralizing antibodies (such as anti-RBD neutralizing antibodies, S1, S2 or S1+S2) are added to corresponding wells and incubated. After plate wash, horseradish peroxidase conjugated anti-human IgG are added followed by incubation.

Following incubation, the plates are washed and added with 3,3′,5,5′-tetramethylbenzidine substrate. The reaction is stopped by adding sulfuric acid. The OD signal values are acquired from each well with a plate reader, and the binding effect is calculated.

B. Evaluation of the ADE Effect of Anti-SARS-CoV-2 Neutralizing Antibodies to FcγR-Expressing Cells Infected with Luciferase-Expressing VSV Pseudotyped with SARS-CoV-2 Spike Protein

Cells expressing FcγRIIA (such as FcγRIIA-expressing CHO-K1 cells) are infected with a luciferase-expressing vesicular stomatitis virus pseudotyped with SARS-CoV-2 spike protein, mixed with or without anti-SARS-CoV-2 RBD neutralizing antibodies. After incubation, luciferase reporter gene assay system is used to determine the luciferase activity. The ADE effect of anti-SARS-CoV-2 neutralizing antibodies to FcγR-expressing cells infected with luciferase-expressing VSV pseudotyped with SARS-CoV-2 spike protein are calculated and assessed.

C. Pseudovirus Neutralization Assay of Anti-SARS-CoV-2 RBD Neutralizing Antibodies to ACE2-Expressing Cells Infected with Luciferase-Expressing Vesicular Stomatitis Virus (VSV) Pseudotyped with SARS-CoV-2 Spike Protein

To elucidate whether there is synergistic effect when the anti-SARS-CoV-2 antibodies neutralize pseudovirus to infect the ACE2-expressing target cells, a pseudovirus neutralization assay may be used.

ACE2-expressing cells (such as CHO-K1 cells expressing ACE2) are infected with a luciferase-expressing vesicular stomatitis virus pseudotyped with SARS-CoV-2 spike protein, mixed with or without anti-SARS-CoV-2 neutralizing antibodies. After incubation, luciferase reporter gene assay system is used to determine the luciferase activity. The pseudovirus neutralization effect of anti-SARS-CoV-2 RBD neutralizing antibodies to ACE2-expressing cells infected with luciferase-expressing vesicular stomatitis virus (VSV) pseudotyped with SARS-CoV-2 spike protein is calculated and evaluated.

### EXAMPLES

The following non-limiting examples are illustrative for the disclosure and are not to be construed as to be in any way limiting for the scope of the invention.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### Example 1. Binding of Anti-SARS-CoV-2 RBD Neutralizing Antibodies to FcγRIIA

To elucidate whether there was synergistic effect when the anti-SARS-CoV-2 antibodies bind to the FcγRIA, a FcγRIIA binding ELISA was performed.

Briefly, 4 μg/mL of FcγRIIA (CD1-H5223, ACRO, China, Human Fc gamma RIIA/CD32a (H167) Protein carrying a His Tag at the C-terminus) was coated onto the 96-well plate. The plate was incubated for 16-24 hours in a refrigerator set at 2-8 C. Following incubation, the plates were washed 3 times with 200 μL PBS containing 0.05% Tween 20 and blocked by 19% BSA in 0.05% PBST for 2 hours. Increasing concentrations (0-20 μg/mL, serial dilutions) of anti-SARS-CoV-2 RBD neutralizing antibodies S1 (SA2-S36, ACRO, China), S2 (SAD-S35, ACRO, China), or S1+S2 (a mixture of SA2-S36:SAD-S35 in 1:1 (v/v)), in assay buffer (PBS containing 0.05% Tween 20 and 1% BSA) were added to corresponding wells (100 μL/well) and incubated at 25° C. for 2 hours on an orbital shaker (Innova 40R. New Brunswick Scientific, USA).

After plate wash, horseradish peroxidase conjugated anti-human IgG (Fab specific antibody A0293, Sigma, China; 650 ng/mL) were added at 100 μL per well followed by incubation for 1-2 hours at 25° C. on an orbital shaker. Following incubation, the plates were washed and 100 μL/well of 3,3′,5,5′-tetramethylbenzidine (TMB, 5120-0053, KPL, China) substrate was added to the plate before the reaction was stopped by 1 M sulfuric acid.

The OD signal values were acquired from each well with a plate reader, subsequently exported to a SoftMax Pro software and plotted against the anti-SARS-CoV-2 antibody concentrations. A 4-parameter fit analysis was then used to estimate the activity of the samples relative to each other.

### Results

As shown in FIG. 1, both anti-SARS-CoV-2 RBD neutralizing antibodies SAD-S35 and SA2-S36 bind to FcγRIIA in a dose-dependent manner. However, when SAD-S35 and SA2-S36 are used together, there is no synergistic binding effect observed.

The results suggest there is no synergistic effect produced by the combination of the two anti-SARS-CoV-2 RBD neutralizing antibodies in binding to FcγRIIA.

### Example 2. ADE Effect of Anti-SARS-CoV-2 RBD Neutralizing Antibodies to FcγRIIA-Expressing Cells Infected with Luciferase-Expressing Vesicular Stomatitis Virus (VSV) Pseudotyped with SARS-CoV-2 Spike Protein

Anti-SARS-CoV-2 RBD neutralizing antibodies S1 (SA2-S36, ACRO, China), S2 (SAD-S35, ACRO. China), or S1+S2 (a mixture of SA2-S36: SAD-S35 in 1:1 (v/v) at a starting concentration of 40 μg/mL were serially diluted in assay buffer (CD CHO culture medium, Gibco Cat #10743029). The serial dilutions of the antibodies were mixed with luciferase-expressing vesicular stomatitis virus (VSV) pseudotyped with SARS-CoV-2 spike protein (80033, Beijing SanYao Science & Technology Development Co., China) at a titration of 1.3×10⁴ TCID₅₀ per well in a volume ratio of 1:1, and the mixture was incubated at 37° C. for about 1 hour.

CHO-K1 cells expressing FcγRIIA 131His (M00598, GenScript. China, CHO-KL/human CD32A 131His Stable Cell Line) were seeded in a 96-well plate at 5×10\hat{ }4 cells per well. Some wells were infected with the mixture of luciferase-expressing VSV pseudotyped with SARS-CoV-2 spike protein and the antibodies at a pre-optimized titration of 650 TCID₅₀ per well.

After 20-24 hours of incubation at 37° C. and 5% CO₂, 100 μL/well of PE Britelite-plus (6016769, Perkin Elmer, China) was added and incubated for 3-5 minutes at RT on a shaker and pipetted up and down for 8-15 times before 100 μL/well of supernatant was transferred to a white plate. The luciferase activity was evaluated by luminescence readout (Envision 2104 Multilabel Reader).

### Results

As shown in FIG. 2, both anti-SARS-CoV-2 RBD neutralizing antibodies SAD-S35 and SA2-S36 could facilitate internalization of pseudovirus into FcγRIIA-expressing CHO-K1 cells. In the presence of both SAD-S35 and SA2-S36, the antibody-dependent enhancement effect is significantly improved. The remarkably increased LUM values for the antibody combination at concentrations of 200, 1000 and 2500 ng/mL showed a synergistic effect.

The results suggest the two anti-SARS-CoV-2 RBD neutralizing produced synergistic effect in binding to FcγRIIA and thus have synergistic ADE effect.

### Example 3. Pseudovirus Neutralization Assay of Anti-SARS-CoV-2 RBD Neutralizing Antibodies to ACE2-Expressing Cells Infected with Luciferase-Expressing Vesicular Stomatitis Virus (VSV) Pseudotyped with SARS-CoV-2 Spike Protein

To elucidate whether there was synergistic effect when the anti-SARS-CoV-2 antibodies neutralize pseudovirus to infect the ACE2-expressing target cells, a pseudovirus neutralization assay was developed.

Anti-SARS-CoV-2 RBD neutralizing antibodies S1 (SA2-S36, ACRO. China). S2 (SAD-S35, ACRO, China), or S1+S2 (a mixture of SA2-S36: SAD-S35 in 1:1 (v/v)) at a starting concentration of 40 μg/mL were serially diluted in assay buffer (CD CHO culture medium, Gibco Cat #10743029). The serial dilutions of the antibodies were mixed with luciferase-expressing vesicular stomatitis virus (VSV) pseudotyped with SARS-CoV-2 spike protein (80033, Beijing SanYao Science & Technology Development Co., China) at a titration of 1.3×10⁴ TCID₅₀ per well in a volume ratio of 2:1, and the mixture was incubated at 37° C. for about 1 hour.

CHO-K1 cells expressing ACE2 (recombinant clone stable CHOK1 cell line constitutively expressing full length of human ACE2) were seeded in a 96-well plate at 5×10⁴ cells per well. Some wells were infected with the mixture of luciferase-expressing VSV pseudotyped with SARS-CoV-2 spike protein and the antibodies at a pre-optimized titration of 650 TCID₅₀ per well.

After 20-28 hours of incubation at 37° C. and 5% CO₂, 100 μL/well of PE Britelite-plus (6016769, Perkin Elmer, China) was added and incubated for 3-5 minutes at RT on a shaker and pipetted up and down for 8-15 times before 100 μL/well of supernatant was transferred to a white plate. The luciferase activity was evaluated by luminescence readout (Envision 2104 Multilabel Reader).

The neutralization inhibition rate (NI %) was calculated using the following formula: PGP-22X

$$NI \% = \frac{1 - ( average LI of the smaple - average LI of blank control )}{average LI of negative control - average LI of blank control} \times 100 \%$$

wherein:
NI=neutralization inhibition rate;
LI=luminous intensity;
blank control=cell only; and
negative control=cell+pseudovirus, without antibody

The data was subsequently exported to a SoftMax Pro software and plotted using 4-parameter fit analysis.

### Results

As shown in FIG. 3, both anti-SARS-CoV-2 RBD neutralizing antibodies SAD-S35 and SA2-S36 could inhibit pseudovirus infection of ACE2-expressing CHO-K1 cells. In the presence of both SAD-S35 and SA2-S36, there is no synergistic effect observed.

The results suggest the two anti-SARS-CoV-2 RBD antibodies do not neutralize pseudovirus in a synergistic manner.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for determining whether two or more antibodies have synergistic antibody-dependent enhancement (ADE) effect in SARS-CoV-2 infection, wherein the method comprises:
contacting a combination of said two or more antibodies with FcR-expressing cells infected with pseudovirus, wherein the pseudovirus is pseudotyped with SARS-CoV-2 antigen; and determining whether the combination increases the internalization of the pseudovirus into the FcR-expressing cells synergistically;
optionally, the method further comprises one or both of the steps of:
determining the binding effect of the combination of the two or more antibodies to FcR (such as by ELISA); and/or
determining the pseudovirus neutralization effect of the combination of the two or more antibodies.

2. The method of claim 1, wherein the antibodies are SARS-CoV-2 neutralizing antibodies, such as FcγR binding SARS-CoV-2 neutralizing antibodies.

3. The method of claim 1, wherein the FcR is a FcγR, for example FcγR selected from FcγRI (e.g., FcγRIa, FcγRIb and FcγRIc), FcγRII (e.g., FcγRIIa, FcγRIIb and FcγRIIc) and FcγRIII (e.g., FcγRIIIa or FcγRIIIb), preferably FcγRII (such as FcγRIIa); and/or
the FcR-expressing cell is selected from the group consisting of natural FcR-expressing cells, such as monocytes, macrophages, dendritic cells, B cells, hematopoietic cells; or engineered cell, such as CHO cells, HEK 293 T cells.

4. The method of claim 1, wherein the pseudovirus is selected from the group consisting of VSV; retrovirus system, such as murine leukemia virus (MLV); lentivirus system, such as HIV-1; adenovirus; or adeno-associated virus (AAV); and/or
wherein the cell further comprises one or more report genes selected from the group consisting of fluorescin (such as luciferase, GFP), β-galactosidase, secreted alkaline phosphatase (SEAP).

5. The method of claim 1, wherein the SARS-CoV-2 antigen is derived from the spike protein (S protein), such as is derived from the RBD region or the N-terminal domain of S protein.

6. The method of claim 1, wherein the method further comprises one or both of the steps selected from the group consisting of:
determining the binding effect of the combination of the two or more antibodies to FcR (such as by ELISA); and/or
determining the pseudovirus neutralization effect of the combination of the two or more antibodies.

7. The method of claim 6, wherein the SARS-CoV-2 antigen is derived from S protein and the pseudovirus neutralization effect is tested with ACE2-expressing cells, such as ACE2-expressing CHO-K1 cells.

8. The method of claim 1, wherein the method is used:
(a) in the selection of antibodies for used in the preparation of a vaccine or a combination of vaccines for preventing SARS-CoV-2 infection; and/or
(b) in predicting and/or preventing a risk of ADE effect in the treatment or vaccination for SARS-CoV-2 infection; and/or
(c) in the quality control during biopharmaceutical product development, manufacturing and/or storage.

9. Use of substance(s) for determining whether two or more antibodies (such as SARS-CoV-2 neutralizing antibodies) have synergistic antibody-dependent enhancement (ADE) effect in SARS-CoV-2 infection in the preparation of a product (such as a kit) used (a) in the selection of antibodies for used in the preparation of a vaccine or a combination of vaccines for preventing SARS-CoV-2 infection; and/or (b) in predicting and/or preventing a risk of ADE effect in the treatment or vaccination for SARS-CoV-2 infection; and/or (c) in the quality control during biopharmaceutical product development, manufacturing and/or storage, wherein the product is used in a method according to any of claims 1-8.

10. A system for determining whether two or more antibodies (such as SARS-CoV-2 neutralizing antibodies) have synergistic antibody-dependent enhancement (ADE) effect in SARS-CoV-2 infection, wherein the system comprises means for carrying out the method of any of claims 1-8.
